(19) United States
(12) Patent Application Publication
(10) Pub. No.: US 2026/0028306 A1
(43) Pub. Date: Jan. 29, 2026

(54) PROCESS FOR PRODUCING METHYLENEBIS(CYCLOHEXYLAMINE)

(71) Applicant: Evonik Operations GmbH, Essen (DE)

(72) Inventors: Christoph HILLER, Dülmen (DE); Laura-Selin HARDING, Dorsten (DE); Johannes NEUKÄUFER, Oer-Erkenschwick (DE); Hendrik RÖSSLER, Düsseldorf (DE); Maria Vargas GÓMEZ, Haltern am See (DE); Florian BOECK, Witten (DE); Tobias WINKLER, Dülmen (DE); Axel HENGSTERMANN, Senden (DE)

(73) Assignee: Evonik Operations GmbH, Essen (DE)

(21) Appl. No.: 19/281,936

(22) Filed: Jul. 28, 2025

(30) Foreign Application Priority Data
Jul. 26, 2024 (EP) 24191091.8

(51) Int. Cl. C07C 209/72; C07C 209/84; C07C 209/86

(52) U.S. Cl. CPC C07C 209/72; C07C 209/84; C07C 209/86

(57) ABSTRACT

The present invention provides a process for producing methylenebis(cyclohexylamine), comprising the steps of 1) catalytically hydrogenating MDA, 2) removing the catalyst and 3) subsequently distilling the product of the hydrogenation, in which at least one dividing-wall column is used in step 3). Exposure to high temperature and residence time of the product is reduced and problems due to crystallization do not occur. The methylenebis(cyclohexylamine) isomer ratio also may remain largely constant during the distillation.

The present invention relates to a process for producing methylenebis(cyclohexylamine).

Methylenebis(cyclohexylamine) is an important industrial chemical that is used in many typical amine reactions, such as reactions with carboxylic acids, phosgene, aldehydes, ketones and epoxides. Methylenebis(cyclohexylamine) allows use to be made of the advantages of cycloaliphatic amines in epoxy systems: low mixed viscosities, moderate reactivity and little exothermic behaviour, as well as outstanding mechanical properties and excellent chemical resistance. By comparison with other amines, the tendency to carbamate formation is reduced, which is an advantage for use as an epoxy curing agent.

Methylenebis(cyclohexylamine) is a cycloaliphatic amine that is usually produced by hydrogenation of diaminodiphenylmethane. Diaminodiphenylmethane, on account of being produced in turn from aniline and formaldehyde, is also referred to as methylenedianiline. The abbreviation MDA, for methylenedianiline, is also often used for diaminodiphenylmethane. Methylenebis(cyclohexylamine) is accordingly also often referred to as H12MDA.

As a consequence of the process by which it is produced, MDA is a mixture of different diaminodiphenylmethanes—primarily 4,4′-diaminodiphenylmethane, but the 2,4′- and 2,2′-isomers may also be present. In addition, reaction products having three or more aromatic rings, in particular ones having three or more phenyl rings, may be present in the mixture. The corresponding reaction products having three or more aromatic rings are also referred to as multiring compounds.

As a consequence of the composition of the MDA used, commercially available methylenebis(cyclohexylamine) is mostly 4,4′-diaminodicyclohexylmethane, or bis(para-aminocyclohexyl)methane. 4,4′-Diaminodicyclohexylmethane can in turn be in the form of trans/trans, cis/cis and cis/trans isomers and is therefore generally a mixture of these isomers in varying proportions. Whereas methylenebis(cyclohexylamine) grades having a low trans/trans content of 4,4′-diaminodicyclohexylmethane (10-30%) are used in the field of amine and isocyanate crosslinkers, especially in the field of 2-component resins, grades having a high trans/trans content (>48%) are mainly used as regulator in polyamide compounds. The potential presence of the corresponding 2,4′-and 2,2′-diaminophenylmethane isomers in the MDA means that 2,4′-diaminodicyclohexylmethane and 2,2′-diaminodicyclohexylmethane may likewise be present in methylenebis(cyclohexylamine). In addition, methylenebis(cyclohexylamine) may additionally contain (sometimes partially) hydrogenated multiring compounds.

There is prior art in which methylenebis(cyclohexylamine) is not the desired main product of a synthesis and is removed therefrom.

U.S. Pat. No. 2,511,028 A discloses the partial hydrogenation of bis(4-aminophenyl)methane to obtain the corresponding product having just one aromatic ring. The methylenebis(cyclohexylamine) can be removed from the desired main product by distillation.

U.S. Pat. No. 4,399,307 A is concerned with the distillative removal of by-products from a product that is structurally similar to methylenebis(cyclohexylamine), namely the diamine 2,2′-bis(4-aminocyclohexyl)propane (PACP). A process for removing a group of organic impurities that includes bis(4-aminocyclohexyl)methane by addition of a solvent and distillation is disclosed.

Processes for producing methylenebis(cyclohexylamine) have long been known in the prior art.

For instance, U.S. Pat. No. 3,742,049 A discloses a process for producing a bis(4-aminocyclohexyl)methane or -ethane in which the corresponding bis(4-nitrophenyl)alkane is hydrogenated in the presence of a ruthenium oxide catalyst. However, the production of the reactants and their handling is laborious.

Processes for producing bis(4-aminocyclohexyl)methane from MDA have likewise long been disclosed in the prior art.

EP 0 639 403 A2 discloses a process for the hydrogenation of MDA in the melt. Purification of the product is not described.

Processes for the hydrogenation of MDA in solution have likewise long been part of the prior art: U.S. Pat. No. 5,214,212 A discloses a process for the catalytic hydrogenation of MDA to bis(4-aminocyclohexyl)methane that, in addition to hydrogen, employs a noble metal catalyst, a promoter and a solvent. In the examples, the product is removed from the reaction mixture by filtration.

US 2002/0183556 A1 discloses the hydrogenation of MDA to methylenebis(cyclohexylamine) in a suspension reactor using a ruthenium catalyst. The solvent optionally present can be removed by distillation.

DE 101 19 135 A1 likewise discloses a process for producing MDA to methylenebis(cyclohexylamine) in a suspension reactor. This can consist of a cascade of two or more interconnected reactors. The solvent optionally present can be removed by distillation.

EP 1 566 372 A1 discloses a process for hydrogenating a substance in a trickle-bed reactor. The substance used may be MDA.

US 2005/0261525 A1 discloses a process for the catalytic hydrogenation of MDA using a lithium aluminate-supported rhodium-and ruthenium-containing catalyst.

EP 3 000 803 A1 discloses a process for producing diaminodicyclohexylmethane in which initially 4,4′-diaminophenylmethane is hydrogenated up to a conversion of 90-98%. Then, instead of pure 4,4′-diaminophenylmethane, a mixture of 2,4′-diaminophenylmethane and 4,4′-diaminophenylmethane is supplied. Once the conversion of this mixture is over 90%, the conversion is continued for several hours further. Thereafter, only 4,4′-diaminophenylmethane is supplied again.

Processes are in addition known from the prior art in which 4,4′-diaminodicyclohexylmethane is produced and then processed by distillation to obtain purer products:

DE 25 02 893 A1 discloses a process for producing cycloaliphatic amines by catalytic hydrogenation of the corresponding aromatic amines in the presence of a ruthenium catalyst. In the examples, 4,4′-diaminodiphenylmethane is hydrogenated in the presence of a catalyst. The reaction product is taken up in methanol, filtered and distilled.

U.S. Pat. No. 3,856,862 A discloses a process for the catalytic hydrogenation of 4,4′-diaminodiphenylmethane to 4,4′-diaminodicyclohexylmethane in the presence of ammonia using a supported rhodium catalyst. In the examples, the resulting methylenebis(cyclohexylamine) is after removal of the catalyst purified by washing with isopropanol and removing the solvent using a micro-Vigreux column. The product contains mostly methylenebis(cyclohexylamine) with small proportions of the partial hydrogenation product H6MDA containing only one cyclohexyl ring that is also obtained, plus other impurities.

U.S. Pat. No. 4,754,070 A discloses a process for the catalytic hydrogenation of 4,4′-diaminodiphenylmethane having an oligomer content of 10-30% in the presence of a catalyst comprising the metals rhodium and ruthenium. In one variant of the example, the solvent-containing product mixture is separated by distillation.

WO 2006/065961 A1 discloses a process for the hydrogenation of MDA alongside at least one second aromatic amine. In the examples, a 1:1 mixture of MDA and aniline is reacted to diaminodicyclohexylmethane and cyclohexylamine in the presence of a ruthenium/aluminium oxide catalyst. The reaction mixture is fractionally distilled.

WO 2009/090179 A2 discloses a process for producing cycloaliphatic amines by hydrogenation of the corresponding aromatic compounds with hydrogen-containing gas in the presence of ruthenium-containing catalysts and in the presence of suspended inorganic additives. The aromatic compound employed may be 4,4′-diaminodiphenylmethane. The mixture obtained after the reaction can be purified by rectification or distillation.

WO 2009/144148 A1 discloses a process for producing aromatic diisocyanates in which aromatic amines are in a substep hydrogenated to cycloaliphatic amines in the presence of a catalyst. Example 2 employs MDA as the aromatic amine. The product is purified by distillation.

WO 2009/153123 A1 discloses a process for the hydrogenation of organic compounds in a multiphase system in the presence of a homogeneous or heterogeneous catalyst, said process being carried out in two stages. A reactant employable with preference is MDA. According to the description, the reaction mixture obtained can be purified by distillation.

EP 2 883 863 A1 discloses a process for the hydrogenation of 4,4′-methylenedianiline with hydrogen in the presence of a catalyst containing ruthenium on a zirconium dioxide support material. The hydrogenation mixtures obtained can be purified in accordance with the process of the invention, for example by distillation.

EP 2 502 900 A1 discloses a process for producing 4,4′-diaminocyclohexylmethane in which MDA is hydrogenated in the presence of an organic solvent and a catalyst and the reaction is stopped when the reaction solution comprises 0-5% by weight of MDA and 1-20% by weight of H6MDA. The 4,4′-diaminocyclohexylmethane obtained is subsequently isolated. In one embodiment, the reaction mixture is filtered so as to recycle the catalyst and return it to the reaction. The filtrate is distilled in a first distillation column and the solvent removed at the top of the column, while a crude H12MDA stream is withdrawn at the base of the column. The crude H12MDA stream undergoes a distillative purification in a second column in which low boilers/non-aminated products are withdrawn at the top of the column and a product stream is withdrawn at the base of the column. Finally, the product stream withdrawn at the lower end of the second column is supplied to a third distillation column and distilled in a manner such that 4,4′-diaminocyclohexylmethane is withdrawn at the top of the column, MDA and H6MDA can be obtained (and recycled) as sidestream products, and secondary amines/high boilers remain at the base of the column.

The disadvantages of the abovementioned processes and especially by comparison with EP 2 502 900 A1 are a greater exposure to high temperature on account of the constructional design with two columns for separation of the product, and an associated longer residence time. It is also disadvantageous in the abovementioned processes that the methylenebis(cyclohexylamine) is condensed at the top of the second column. The higher melting point of particular isomers of methylenebis(cyclohexylamine) means they crystallize to some degree, which necessitates temporarily decommissioning the column and thus reduces operating times, or alternatively requires the use of two condensers connected in parallel, which however involves considerable complexity and gives rise to problems with operability.

Finally, it would in addition be desirable if the methylenebis(cyclohexylamine) isomer ratio were to remain constant in the distillation.

It is accordingly an object of the present invention to overcome the disadvantages of the prior art. More particularly, it is an object of the present invention to provide a process for the production and distillative purification of methylenebis(cyclohexylamine) in which the exposure to high temperature and residence time of the product is reduced and problems due to crystallization do not occur. An additional object of the present invention is that the methylenebis(cyclohexylamine) isomer ratio remains largely constant during the distillation.

This object is achieved by the process of the invention for producing methylenebis(cyclohexylamine), comprising the steps of:
1) catalytically hydrogenating MDA,
2) removing the catalyst and
3) subsequently distilling the product of the hydrogenation, wherein at least one dividing-wall column is employed in the distillation in step 3).

The energy consumption for the separation process of the invention is advantageously 450 to 500 kilojoules per kilogram of methylenebis(cyclohexylamine). The amount of energy needed for two interconnected columns is on the other hand between 550 and 650 kilojoules per kilogram of methylenebis(cyclohexylamine).

### Step 1)—Catalytic Hydrogenation

Step 1) is the catalytic hydrogenation of MDA to methylenebis(cyclohexylamine) in the presence of a catalyst.

The catalysts employed may in principle be any catalysts that catalyse the hydrogenation of phenyl groups. In principle, these may be homogeneous or heterogeneous catalysts. Heterogeneous catalysts are however preferred.

In particular, catalysts containing active metals selected from nickel, cobalt, palladium, platinum, ruthenium and/or rhodium have been found to be particularly suitable.

To increase activity, selectivity and/or service life, the catalysts may additionally comprise or have been treated with doping metals or modifiers. Preferred doping metals may be selected from the group consisting of Mo, Fe, Ag, Cr, V, Ga, In, Bi, Ti, Zr, Mn and the rare earths. Preferred modifiers are ones that allow the acid-base properties of the catalysts to be influenced, in particular alkali metals, alkaline earth metals, phosphoric acid and sulfuric acid, and compounds and salts thereof.

The catalysts can preferably be used in the form of powders or shaped bodies, for example extrudates or compressed powders. It is possible to employ unsupported catalysts, Raney-type catalysts or supported catalysts.

Preferred support materials for supported catalysts are activated carbons and inorganic oxides, particularly Al₂O₃, SiO₂, TiO₂, ZrO₂, ZnO and MgO, and also bentonites, aluminosilicates, kaolins, clays, kieselguhrs and lithium aluminates. The active metal can be applied to the support material in a manner known to those skilled in the art, for example by impregnation, spray application or precipitation. Depending on the method of catalyst production, further preparation steps known to those skilled in the art are necessary, for example drying, calcining, shaping and activation. For shaping, it is optionally possible to add further auxiliaries, for example graphite or magnesium stearate.

Preference is given to using supported catalysts containing ruthenium, rhodium or Rh/Ru combinations as essential active metals. Preferred support materials are ones based on Al₂O₃, SiO₂, TiO₂ and ZrO₂. Very particular preference is given to Al₂O₃ and SiO₂.

Preference is given to using catalysts known to be employable for production of a methylenebis(cyclohexylamine) having a trans/trans content of the 4,4′-isomer of between 10% and 30%, in particular between 15% and 25%. Such catalysts are described for example in documents EP 1 366 812 A1, EP 0 066 211 A1, DE 100 54 347 A1, EP 0 392 435 A1, EP 0 630 882 A1, EP 0 639 403 A2 and U.S. Pat. No. 5,545,756 A. Very particularly preferably, the hydrogenation is carried out in the presence of a supported catalyst that contains, applied on a support, active metal in an amount of 0.01% to 20% by weight based on the supported catalyst, and the active metal thereof is ruthenium alone or ruthenium and at least one metal of subgroups I, VII or VIII [groups 7-11] of the periodic table of the elements. This catalyst allows particularly low trans/trans contents of 4,4′-diaminodicyclohexylmethane to be achieved.

In order to be able to selectively and reproducibly produce a methylenebis(cyclohexylamine) having a particular trans/trans content of the 4,4′ isomer, it is additionally advantageous to precisely control the temperature, conversion and residence time in the reactor. The hydrogenation is for this reason preferably carried out in continuously operated reactors, since these permit corresponding control. Suitable reactors for continuous hydrogenation are familiar to those skilled in the art. In a preferred embodiment of the invention, the continuous hydrogenation of MDA is carried out in fixed-bed reactors.

The reaction is preferably carried out in two or more reaction spaces connected in series. The main advantage of this reaction regimen is that the reaction spaces can be heated or cooled independently of one other, thus providing better options for controlling the content of trans/trans-4,4′-H12MDA. A further advantage is that a decline in catalyst activity can be compensated more selectively by a temperature adjustment, and partial replacement of the catalyst is possible when needed. The separate reaction spaces can be realized for example by two or more fixed-bed reactors connected in series, for example tube-bundle reactors and/or shaft furnaces. Another option is to house, within one reactor, catalyst beds that are spatially separate from one another and can be heated or cooled. The fixed-bed reactors can be operated in a liquid-phase mode, but a trickle-bed mode is preferred. Preferred continuous suspension reactors are tubular and bubble column reactors.

The LHSV value is preferably in the range from 0.01 to 1 h⁻¹ (l of aromatic amine to be hydrogenated per l of fixed-bed catalyst and hour).

The hydrogenation is in addition preferably carried out at temperatures in the range from 50 to 200° C., preferably between 80 and 170° C. The hydrogen pressure is preferably between 1 and 30 MPa, preferably between 5 and 15 MPa.

A solvent can in principle be present in the hydrogenation, but does not have to be. It is however preferable to hydrogenate MDA in a solvent. The proportion of the solvent is further preferably between 10% and 90%, even further preferably between 50% and 90%, based on the mass of the solution. Preferred solvents can be selected from the group consisting of primary, secondary and tertiary monohydric or polyhydric alcohols (in particular methanol, ethanol, n-and i-propanol, 1-, 2-, i- and tert-butanol, ethylene glycol and ethylene glycol mono (C1-C3) alkyl ethers), linear ethers (in particular ethylene glycol di(C1-C3) alkyl ethers) and MTBE, cyclic ethers (in particular tetrahydrofuran and dioxane) and alkanes (in particular n-and isoalkanes having 4-12 carbon atoms, further preferably n-pentane, n-hexane and isooctane, and cyclic alkanes, further preferably cyclohexane and decalin). Whereas alcohols can result in alkylation of the amino groups, ethers do not have this disadvantage and are thus particularly preferred. A very particularly preferred solvent is tetrahydrofuran.

However, solvent can also likewise preferably be the hydrogenation product itself.

The hydrogenation can preferably also be carried out in the presence of ammonia, a primary, secondary or tertiary amine, or a polycyclic amine having a bridging nitrogen atom.

Particular preference is given to using as the mixture to be hydrogenated an MDA that comprises at least 70% by weight of 4,4′-diaminodiphenylmethane and 0.01% to 2% by weight of N-methyl compounds, in each case based on the total mass of compounds having aromatic rings. Even further preference is given to using an MDA consisting of 74-85% by weight of 4,4′-MDA, 3-20% by weight of 2,4′-MDA, less than 1% by weight of 2,2′-MDA and up to 1% by weight of N-methyl compounds. With the cited mixtures, a hydrogenation mixture can be particularly readily obtained that has a trans/trans content of the 4,4′ isomers of 10% to 30%.

### Step 2)—Removal of the Catalyst

In step 2), the catalyst is removed. When the catalyst is a fixed-bed catalyst, this is achieved by discharging the hydrogenated product from the fixed-bed reactor. When the catalyst is a heterogeneous catalyst that is not a fixed-bed catalyst, this can preferably be done by decanting, centrifuging or filtering. The catalyst is more preferably removed by filtration, for which the use of screen filters, cartridge filters or crossflow filters is very particularly preferred.

In addition, any solvent(s) used can be removed before or after removal of the catalyst. Further preferably, the removal of solvent(s) takes place after the catalyst has been separated off.

Preference is given to removing the solvent by distillation. Further preferably, this can be done through the use of a distillation column. In order to further reduce a subsequent accumulation of solvent(s), a purge step can also be included.

### Step 3)—Distillation of the Product of the Hydrogenation

After the catalyst and any solvent(s) have been removed, the resulting crude product of the hydrogenation is distilled. At least one dividing-wall column is used here.

The distillation of the hydrogenation product comprising methylenebis(cyclohexylamine) can thus involve one, two or more than two dividing-wall columns.

It is however preferable that the product of the hydrogenation is distilled exclusively through the use of a dividing-wall column, since this ensures a particularly low product residence time in the distillation while maintaining adequate separation and the energetic optimum is achieved.

Upstream or downstream of the distillation of the hydrogenation product by means of at least one dividing-wall column it is additionally possible to provide, for distillative separation, at least one column that is not a dividing-wall column. However, this is preferably omitted, since the separating effect of the at least one dividing-wall column is already sufficient.

Further preferably, the at least one dividing-wall column is operated in a manner such that the product methylenebis(cyclohexylamine) is discharged at the side of the dividing-wall column. This prevents crystallization particularly efficiently, since the product does not come into contact with the condenser. The takeoff preferably occurs in liquid form.

Very particularly preferably, the distillation of the hydrogenation product in step 3) is carried out exclusively through the use of a dividing-wall column.

The construction of dividing-wall columns is known to those skilled in the art. Unlike other columns, these have a dividing wall present in the column, which is as far as possible vertically disposed.

The dividing wall can here in principle be in any position within the dividing-wall column. For example, it is possible that i) the dividing wall extends from a dividing-wall-free bottoms region up to a dividing-wall-free top region, ii) the dividing wall closes off the lower end of the column, but not the top region, or iii) the dividing wall closes off the top region, but not the lower end of the column.

Preferably, the dividing wall extends from a dividing-wall-free bottoms region up to a dividing-wall-free top region. Even more preferably, the dividing wall of the at least one dividing-wall column, preferably of the dividing-wall column, is continuously present at a height of 15 to 80%, further preferably at a height of 20 to 75%, particularly preferably at a height of 25 to 75%, of the dividing-wall column, measured from the lower end of the column. The “height” of the dividing-wall column is to be understood as meaning the distance from the base of the column to the column top.

The dividing wall is normally located in the middle of the column. However, in order to achieve advantageous properties it is also possible for the dividing wall to have an offset towards the inflow or towards the sidestream takeoff. Preferably, the offset of the dividing wall has a value of between 0 and 30% of the diameter from the middle towards the sidestream region or inflow region. This value is further preferably between 0 and 20%.

To achieve advantageous results, the dividing wall may also be designed as an asymmetric dividing wall. An asymmetrically designed dividing wall has a horizontal offset. Further preferably, the horizontal offset is in this case designed such that the lower end of the dividing wall is closer to the sidestream takeoff than the upper end. As a consequence, the cross-sectional area below the sidestream takeoff is reduced there, whereas it is increased in the upper region on the side of the inflow. The advantage of this design is optimization of the liquid load and gas distribution and thus of the pressure drop of the column. Further preferably, the offset of the dividing wall above the inflow is 2 to 30% of the cross-sectional area of the column and the offset of the dividing wall below the sidestream is between 2 and 40% of the cross-sectional area of the column. This can have a particularly favourable effect on quality, yield and energy requirements.

Preferably, the distillation with the at least one dividing-wall column, further preferably the dividing-wall column, is operated so as to avoid crystallization, such that
a) the inflow is located at the height of at least a portion of the dividing wall;
b) the sidestream takeoff for drawing off purified methylenebis(cyclohexylamine) is located at the height of at least a portion of the dividing wall, on the other side of the dividing wall from the inflow;
c) the distillate is withdrawn at the top of the dividing-wall column and partly discharged and partly returned to the dividing-wall column and
d) the bottoms located at the base of the dividing-wall column is discharged.

The methylenebis(cyclohexylamine) withdrawn in step b) is the desired product of the process here. The distillate withdrawn in c) comprises low boilers and any solvent(s) present. Present in the bottoms are high boilers, which can include multiring compounds.

Further preferably, the distillate is withdrawn in step c) such that
the distillate is withdrawn at the top of the dividing-wall column,
it undergoes at least partial condensation in a connected condenser,
and at least a portion of the condensate is returned to the top region of the dividing-wall column, and a low boilers stream possibly still present in volatile form downstream of the condenser is discharged together with non-returned condensate.

Preferably, the inflow of the at least one dividing-wall column, preferably of the one dividing-wall column, is present at a height of 25 to 66%, further preferably at a height of 30 to 60%, even more preferably at a height of 40 to 60%, of the height of the column, measured from the lower end of the column.

The at least one dividing-wall column, preferably the dividing-wall column, further preferably has a number of theoretical plates of from 10 to 90, further preferably from 15 to 80, further preferably from 20to 70.

The at least one dividing-wall column, preferably the dividing-wall column, is preferably operated at a bottoms temperature of from 190 to 300° C., further preferably from 190 to 270° C., very particularly preferably from 190 to 240° C., since this affords products achieving particularly good qualities (especially having particularly low coloration) and yields and with particularly low energy requirements. The pressure is preferably 1 to 30 mbar, further preferably 2 to 20 mbar and very particularly preferably 3 to 15 mbar.

The at least one dividing-wall column, preferably the dividing-wall column, is preferably operated with a liquid distribution of the liquid flowing down above the dividing-wall column onto the two sides in a ratio between 10/90 and 40/60 (inflow part/sidestream part), preferably between 15/85 and 30/70.

A particularly good separation of the hydrogenation product, i.e. a particularly pure methylenebis(cyclohexylamine), can be obtained with a liquid load of less than or equal to 1.5 m³/(m²h) below the sidestream takeoff and above the bottoms and above the lower end of the dividing wall, further preferably between 0.1 and 1 m³/(m²h), since this allows the separation to be operated at the energetic optimum. This low liquid load and the requisite division between the two regions (inflow part and sidestream part) represents a particularly low case for the dividing-wall column design, whereby the operability increasingly declines, particularly as a result of the hydraulic coupling of the liquid flows of a dividing-wall column. In an energetically optimal mode of operation, the liquid added to the sidestream region above the dividing wall is largely removed through the sidestream. If the liquid load below the sidestream is consequently operated at too low a level, fluctuations in the liquid distribution and sidestream takeoff can no longer be compensated and the separation performance collapses.

In addition to the lower liquid load, a very low pressure drop between the column top and bottoms is desirable, since a pressure drop that is too high leads to higher pressures in the bottoms and thus to higher boiling temperatures of the components present in the bottoms. Very high bottoms temperatures in turn lead to two practical problems. Firstly, at 255° C. and above there may be detectable decomposition of the bottoms components, as a result of which the specification for the sidestream product methylenebis(cyclohexylamine) is no longer achievable. Secondly, heating the bottoms becomes more difficult, since steam heating is no longer possible at the temperatures that are then reached.

The sidestream takeoff of the at least one dividing-wall column, preferably the one dividing-wall column, is located preferably at a height of 30 to 60%, further preferably at a height of 40 to 60%, even further preferably at a height of 45 to 55%, of the height of the column, measured from the lower end of the column.

The choice of packings is of great importance for the performance of the distillation, since the pressure drop for this operation should preferably be less than 0.2 mbar per theoretical plate. The combination of low liquid load, low pressure drop, and the associated properties that a packing must fulfil might suggest that the use of a dividing-wall column is not preferable. Surprisingly, it was found to be possible to operate a dividing-wall column in spite of these considerable challenges and despite the fine separation of methylenebis(cyclohexylamine) that is required.

The packing beds of the dividing-wall column are preferably designed to be between 2 and 7 metres in height. Further preferably, they separate liquids very finely such that the maldistribution that occurs within the packing beds is acceptable in terms of separation.

Packing types used are preferably ordered and/or unordered packings, further preferably sheet metal packings and/or fabric packings. These achieve a particularly high separation performance at low pressure drop and low liquid load.

In order to demonstrate the product quality and the validity of the dividing-wall column for separating methylenebis(cyclohexylamine) and the low-and high-boiling secondary components, experimental investigations were carried out.

### BRIEF DESCRIPTION OF DRAWINGS

FIG. 1 shows a variant of the dividing wall which achieve favourable effects.

FIG. 2 shows another variant of the dividing wall which achieve favourable effects.

FIG. 3 shows another variant of the dividing wall which achieve favourable effects.

FIG. 4 shows another variant of the dividing wall which achieve favourable effects.

FIG. 5 shows another variant of the dividing wall which achieve favourable effects.

FIG. 6 shows a schematic design for the entire process.

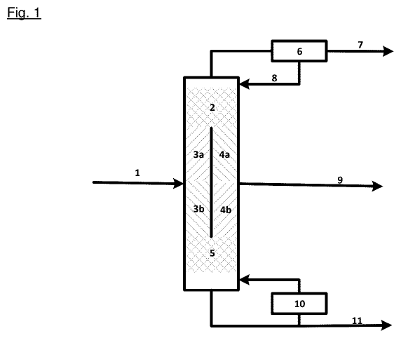

These found that certain executions of the dividing wall achieve favourable effects. The various variants are depicted in FIGS. 1, 3, 4 and 5. The executions of the wall in FIGS. 1, 3, 4 can also be found in patent specification EP 2 569 274 B1. In contrast to the process in said patent, the process of the invention is characterized by a lower operating pressure, the consequently altered gas velocities and the low pressure drop in the column.

Particularly preferably, the process of the invention is a process using a dividing-wall column having at least one dividing wall with one of the following executions:
a) The dividing wall may preferably extend from a dividing-wall-free bottoms region up to a dividing-wall-free top region. In this case, the feedstocks are able to pass across the entire cross section both in the top region and in the bottoms region. Further preferably, the dividing wall runs continuously from 10% to 90% of the height of the column, in each case measured from the lower end of the column. Further preferably, the dividing wall runs continuously from 20 to 80% of the height of the column, even more preferably from 25 to 75%. The position of the dividing wall in the regions mentioned above permits particularly efficient operation of the column with fulfilment of the high separation challenges.

The dividing wall is normally located in the middle of the column. To achieve advantageous properties, the dividing wall can in this execution also have an offset towards the inflow or towards the sidestream takeoff. Preferably, the offset of the dividing wall has a value of between 0 and 30% of the diameter from the centre towards the sidestream region (regions 4a, 4b in FIG. 1) or towards the inflow region (regions 3a, 3b in FIG. 1). This value is further preferably between 0 and 20%.

The inflow is further preferably located on one side of the dividing wall at a height between 25 and 66% of the height of the column, measured from the lower end of the column. Further preferably, above and below the inflow there are between 2 and 50 theoretical plates that are separated from the other side of the dividing wall.

The sidestream is in this execution preferably removed on the other side of the dividing wall at a height of 30 to 60%, further preferably at a height of 40 to 60%, even further preferably at a height of 45 to 55%, of the column height, measured from the lower end.

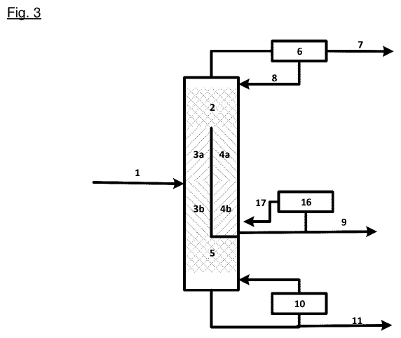

The dividing-wall column designed in this way is a particularly good solution for the purification of methylenebis(cyclohexylamine) that those skilled in the art had not anticipated for the specified considerations of the separation challenges and energy requirements of the requisite pressure drop, at particularly low temperatures, and with achievement of particularly high purity. This configuration makes it possible to separate low, medium and high boilers using just one condenser and just one evaporator.
b) To achieve advantageous results, the dividing wall can also be designed such that the dividing wall closes off the lower end of the column but not the top region. In this case, the dividing wall preferably closes off the part of the column on the side of the sidestream takeoff (FIG. 3) or extends as far as the base of the bottoms.

The dividing wall is normally located in the middle of the column. To achieve advantageous properties, the dividing wall can in this execution also have an offset towards the inflow and towards the sidestream takeoff. Preferably, the offset of the dividing wall has a value of between 0 and 30% of the diameter from the middle towards the sidestream region or inflow region. This value is further preferably between 0 and 20%.

The inflow is further preferably located on one side of the dividing wall at a height between 25 and 66% of the height of the column, measured from the lower end of the column. Further preferably, above and below the inflow there are between 2 and 50 theoretical plates that are separated from the other side of the dividing wall.

The sidestream is in this execution further preferably removed on the other side of the dividing wall at a height between 30 and 60% of the column height, measured from the lower end.

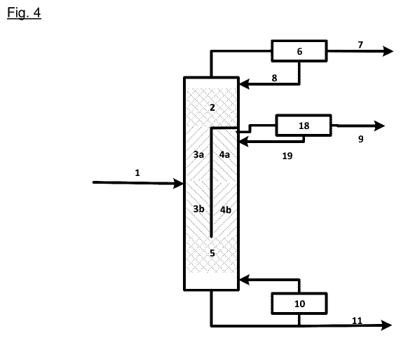

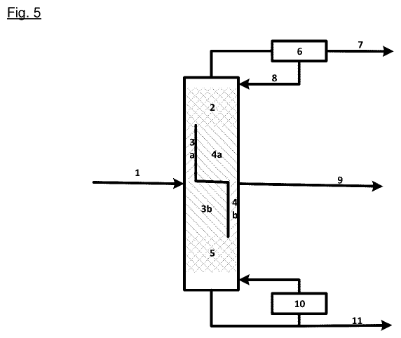

The sidestream is preferably removed at the lower level of the sidestream region of the dividing wall than complete removal. Further preferably, a portion of the stream is transferred again to the gas phase by an evaporator and reintroduced at the same level as the sidestream takeoff. Where the dividing wall continues all the way to the bottom, this results in two independent bottoms. Preferably, the bottoms discharge is implemented with two evaporators and with plates, which in a) are present below the dividing wall across the whole diameter of the column, in equal number on either side. The sidestream is in this case removed as a second bottoms stream.
c) To achieve advantageous results, the dividing wall can also be designed such that the dividing wall closes off the top region, but not the lower end of the column. Preferably, the dividing wall constitutes a barrier wall to the dividing-wall-free top region (FIG. 4) or a dividing wall that continues all the way to the top. The modifications in this concept too are similar to those in b). The sidestream is however preferably removed not as a second bottoms stream or in liquid form above the barrier wall, but as a second distillate or in condensed form below the barrier wall. In this concept, a second condenser is necessary. An advantage is that, above the dividing wall, no low boilers can pass into the sidestream region of the dividing-wall column and contaminate the sidestream.
d) To achieve advantageous results, the dividing wall may also be designed as an asymmetric dividing wall (FIG. 5). An asymmetrically designed dividing wall has a horizontal offset. Further preferably, the horizontal offset is in this case designed such that the lower end of the dividing wall is closer to the sidestream takeoff than the upper end. As a consequence, the cross-sectional area below the sidestream takeoff is reduced there, whereas it is increased in the upper region on the side of the inflow. The advantage of this design is that the liquid load and gas distribution, and thus the pressure drop of the column, is optimized. Further preferably, the offset of the dividing wall above the inflow is 2 to 30% of the cross-sectional area of the column and the offset of the dividing wall below the sidestream is between 2 and 40% of the cross-sectional area of the column. This can have a particularly favourable effect on quality, yield and energy requirements.

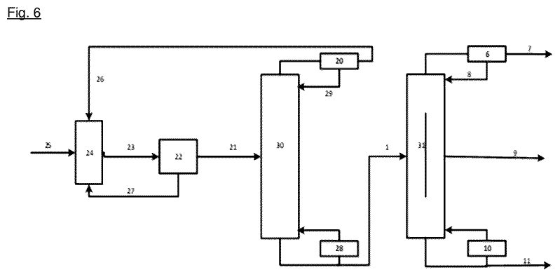

FIG. 6 shows a preferred schematic design for the entire process, beginning with the reaction (24), subsequent catalyst removal (22), solvent removal (30) and subsequent purification in the dividing-wall column (31).

### EXAMPLES

### Example 1

In order to demonstrate the feasibility of separation in a dividing-wall column, experiments were carried out on a pilot scale. For this, a column having a diameter of 150 mm was operated that in the middle region was divided into two columns each having a diameter of 100 mm. The column had a total height of 19 metres, 9 metres of which were filled with a 500 m²/m³ fabric packing. The bottoms was made of metal and comprised a bottoms circuit and an evaporator (10).

The column built onto the bottoms was made of glass. A metal condenser (6) was built onto the glass column.

The glass column was subdivided into four regions: in the upper region (2) the column had two metres of fabric packing, in the inflow region (3a and 3b) four metres of fabric packing (the inflow was loaded in the middle of the inflow region), in the sidestream region four metres of packing (4a and 4b) and in the lower region (5) two metres of fabric packing. The distillate was partly returned to the column as a return flow (8) and partly discharged as a low boilers stream (7). The sidestream (9) was removed in the middle of region 4. The bottoms stream (11) was removed from the bottoms circuit.

### Procedure

Methylenebis(cyclohexylamine) from the hydrogenation of MDA was after the removal of solvent separated with the above setup (see also FIG. 1). For this, in pilot tests 12 kg/h of methylenebis(cyclohexylamine) was loaded in the middle of the prefractionation region (3a and 3b) on an existing column. The temperature of the liquid inflow was here 170° C. The pressure at the top of the column was set at 7 to 10 mbar. The condensed vapour was partly discharged as a distillate discharge and partly returned to the column as a return flow. The amount of liquid obtained above the dividing wall was distributed between the two sides in a ratio of 25% (inflow region, above 3a) and 75% (sidestream region, above 4a). In the middle of the sidestream region (4a, 4b), the purified methylenebis(cyclohexylamine) is removed in purities of over 99.3%.

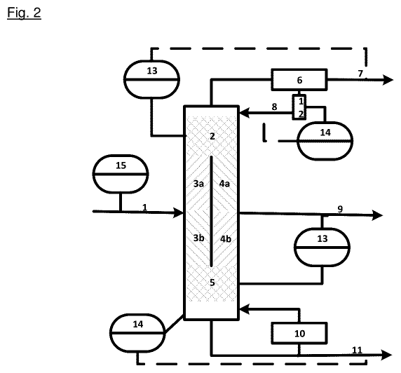

The bottoms flow was taken from the bottoms circuit so as to minimize losses of methylenebis(cyclohexylamine). The product streams and purities thereof are controlled by means of a closed-loop control concept (FIG. 2) that makes appropriate adjustments to the streams via filling level, temperature and flow controls.

### Example 2

As an alternative to the experimental conditions in the previous experiment, the column was operated with a liquid split of 40% inflow region (3a, 3b) and 60% sidestream region (4a, 4b). In the event of changes to the inflow composition, the requirements for the liquid split between the two sides change and this must be altered in order to ensure an optimal separation, taking into consideration the energetic optimum.

### Example 3

As an additional alternative, the load in the column was increased (about 40%) via a higher inflow stream (16.6 kg/h) but with the same liquid split as in experiment 1. The amount of energy was adhered to approximately here.

The results have shown that a high methylenebis(cyclohexylamine) purity can be achieved and at the same time a very high yield, with low losses in the bottoms stream and distillate streams.

The design as a glass column meant it was possible to look into the column during and after the experiments: no contaminants were detected.

Table 1 below summarizes the operating parameters and results for examples 1-3:

TABLE 1

| No. | — | 1 | 2 | 3 |
|---|---|---|---|---|
| Top pressure | mbar, a | 7 | 9 | 8 |
| Bottoms pressure | mbar, a | 13 | 18 | 16 |
| Top temperature | ° C. | 127.5 | 140.7 | 138.5 |
| Bottoms temperature | ° C. | 223.6 | 225.5 | 226.2 |
| Liquid split | — | 25/75 | 40/60 | 25/75 |
| Return flow | kg/h | 13.69 | 21.39 | 18.87 |
| Inflow temperature | ° C. | 170 | 170 | 170 |
| Inflow | kg/h | 11.94 | 11.95 | 16.63 |
| Distillate | kg/h | 0.11 | 0.13 | 0.18 |
| Sidestream | kg/h | 10.00 | 9.91 | 13.94 |
| Bottoms | kg/h | 1.83 | 1.91 | 2.51 |
| Methylenebis(cyclohexylamine) | wt % | 11.92 | 20.84 | 22.07 |
| in distillate | | | | |
| Methylenebis(cyclohexylamine) | wt % | 99.49 | 99.43 | 99.09 |
| in sidestream | | | | |
| Methylenebis(cyclohexylamine) | wt % | 6.77 | 11.85 | 9.20 |
| in bottoms | | | | |
| Energy per kg | kJ/kg | 461 | 763 | 491 |
| methylenebis(cyclohexylamine) | | | | |
| in sidestream | | | | |

### LIST OF REFERENCE NUMERALS

1—Inflow
2—Upper region/top
3—Inflow region, prefractionation region, a above inflow, b below inflow
4—Sidestream region, a above sidestream takeoff, b below sidestream takeoff
5—Lower region/bottoms
6—Condenser
7—Low boilers stream
8—Return flow
9—Sidestream takeoff
10—Evaporator
11—Bottoms stream
12—Distillate vessel
13—Temperature control
14—Filling level control
15—Flow control
16—Sidestream evaporator
17—Sidestream evaporator recirculation
18—Sidestream condenser
19—Sidestream return flow
20—Condenser/solvent column
21—Inflow/solvent column
22—Catalyst separator
23—Reaction product
24—Reactor
25—Reaction reactants
26—Solvent recirculation
27—Catalyst recirculation
28—Evaporator/solvent column
29—Return flow/solvent column
30—Solvent removal (column)
31—Dividing-wall column

1. A process for producing methylenebis(cyclohexylamine), comprising the steps of
1. catalytically hydrogenating MDA,
2. removing the catalyst and
3. subsequently distilling the product of the hydrogenation,
wherein at least one dividing-wall column is employed in the distillation in step 3).

2. The process according to claim 1,
wherein;
a supported catalyst is used as catalyst during said catalytically hydrogenating MDA, said supported catalyst contains, an active metal in an amount of 0.01% to 20% by weight based on the supported catalyst, and wherein the active metal is ruthenium alone or ruthenium and at least one metal of subgroups I, VII or VIII of the periodic table of the elements.

3. The process according to claim 1,
wherein the MDA comprises at least 70% by weight of 4,4′-diaminodiphenylmethane and 0.01% to 2% by weight of N-methyl compounds, in each case based on the total mass of compounds having aromatic rings.

4. The process according to claim 1,
wherein the distillation of the hydrogenation product in step 3) is carried out exclusively with a dividing-wall column.

5. The process according to claim 1,
wherein the dividing wall of the at least one dividing-wall column is continuously present at a height of 15 to 80% of the dividing-wall column, measured from the lower end.

6. The process according to claim 1,
wherein the dividing wall of the at least one dividing-wall column has an offset of 0 to 30% of the diameter from the middle towards the sidestream region or inflow region.

7. The Process according to claim 1,
wherein the dividing wall of the at least one dividing-wall column is designed as an asymmetric dividing wall.

8. The process according to claim 1,
wherein the distillation with at least one dividing-wall column in step 3) is operated such that:
a) the inflow is located at the height of at least a portion of the dividing wall;
b) the sidestream takeoff for drawing off purified methylenebis(cyclohexylamine) is located at the height of at least a portion of the dividing wall, on the other side of the dividing wall from the inflow;
c) the distillate is withdrawn at the top of the dividing-wall column and partly discharged and partly returned to the dividing-wall column and
d) the bottoms located at the base of the dividing-wall column is discharged.

9. The process according to claim 8,
wherein the distillate is withdrawn in step c) such that:
the distillate is withdrawn at the top of the dividing-wall column,
it undergoes at least partial condensation in a connected condenser,
and at least a portion of the condensate is returned to the top region of the dividing-wall column, and a low boilers stream possibly still present in volatile form downstream of the condenser is discharged together with non-returned condensate.

10. The process according to claim 1, wherein the inflow of the at least one dividing-wall column is present at a height of 25 to 66% of the height of the column, measured from the lower end.

11. The process according to claim 1, wherein the at least one dividing-wall column has a number of theoretical plates of from 10 to 90.

12. The process according to claim 1, wherein the at least one dividing-wall column is operated at a bottoms temperature of from 190 to 300° C.

13. The process according to claim 1, wherein the liquid load below the sidestream takeoff and above the bottoms and above the lower end of the dividing wall is less than or equal to 1.5 m³/(m²h).

14. Process according to claim 1, wherein the sidestream takeoff of the at least one dividing-wall column is located at a height of 30 to 60% of the column, measured from the lower end.

15. Process according to claim 1, wherein the packing beds of the at least one dividing-wall column are 2 to 7 metres in height.
